(19) United States
(12) Patent Application Publication
(10) Pub. No.: US 2024/0241138 A1
(43) Pub. Date: Jul. 18, 2024

(54) Blood Test to Screen Out Parkinson's Disease

(71) Applicant: UNIVERSITY OF NORTH TEXAS HEALTH SCIENCE CENTER AT FORT WORTH, Fort Worth, TX (US)

(72) Inventor: O'Bryant Sid E., Aledo, TX (US)

(21) Appl. No.: 18/559,096

(22) PCT Filed: May 6, 2022
(86) PCT No.: PCT/US2022/028092

§ 371 Date: Nov. 6, 2023

Related U.S. Application Data
(60) Provisional application No. 63/185,563, filed on May 7, 2021.

(51) Int. Cl. G01N 33/68; G01N 21/76; G16H 50/20

(52) U.S. Cl. CPC G01N 33/6896; G01N 21/76; G16H 50/20; G01N 2333/4713; G01N 2333/4737; G01N 2333/524; G01N 2333/525; G01N 2333/5409; G01N 2333/5412; G01N 2333/5418; G01N 2333/5428; G01N 2333/70525; G01N 2333/7151; G01N 2333/745; G01N 2800/2835

(57) ABSTRACT

In one aspect, the present disclosure relates to a method for excluding a subject from the need for diagnostic testing for Parkinson's disease (PD). In another aspect, the present disclosure relates to a method for excluding a subject from recruitment into a clinical study for an investigational PD medication. In yet another aspect, the present disclosure relates to a method for screening a subject to determine whether the subject is ruled out as having PD, wherein the subjects who cannot be ruled out are administered a diagnostic test for PD, a treatment for PD, or a combination thereof.

### CROSS-REFERENCE TO RELATED APPLICATIONS

This application claims priority under 35 U.S.C. § 119(e) to U.S. Provisional Patent Application 63/185,563, filed May 7, 2021, the disclosure of which is incorporated herein by reference in its entirety.

### STATEMENT OF FEDERALLY FUNDED RESEARCH

This invention was made with government support under AG054073 and AG058537 awarded by the National Institutes of Health. The government has certain rights in the invention.

### BACKGROUND OF THE INVENTION

Parkinson's disease (PD) is the second most common neurodegenerative disease affecting over 1% of individuals over the age of 65 in the United States (U.S). The cost of PD to society was reported to be $23 billion annually in the U.S. in 2005. Given the rapidly growing segment of the elderly population, these costs will continue to increase over the next several decades. The most accurate diagnosis of PD comes from specialty clinics where clinical assessments and advanced neurodiagnostic procedures are costly, time-consuming, and invasive. In the U.S., primary care clinics serve as the “gatekeeper” to specialty clinics and these front-line primary care practitioners provide the referrals for advanced diagnostic procedures. However, the average duration of primary care visits is around 18 minutes, making detailed neurological examinations difficult.

In 2017, community-dwelling PD patients and general practitioners (GPs) were interviewed to understand their thoughts on the role of primary care in PD management. Discrepancies between patients' and GP views were found as patients felt that GPs lacked expert knowledge or skills and diminished the role of GPs in patients at advanced PD stages. GPs, on the other hand, valued patient autonomy in early-stage decision making but in more advanced PD stages felt a more active role of the GP is warranted. One conclusion of the study was that patients would likely benefit from the more holistic approach brought by the GP if done in conjunction with specialty care. Currently, however, there are no rapid or cost-effective screening tools for primary care providers to use in daily practice to rule out PD in patients and therefore prevent further diagnostic testing for PD.

There is thus a need in the art for a more rapid and cost-effective method to screen a subject to rule out PD and to prevent the subject from undergoing more invasive and/or expensive testing for Parkinson's disease. The present disclosure satisfies this unmet need.

### SUMMARY OF THE DISCLOSURE

In one aspect, the present disclosure provides a method for excluding a subject from the need for diagnostic testing for Parkinson's disease, the method comprising: (a) obtaining a blood, plasma, or serum sample from the subject; (b) measuring in the blood, plasma, or serum sample the expression level of one or more biomarkers selected from the group consisting of: interleukin (IL)-7, tumor necrosis factor alpha (TNFα), IL-5, IL-6, C-reactive protein (CRP), IL-10, soluble intracellular adhesion molecule (sICAM-1), Factor VII, 1309, alpha-2-microglobulin (A2M), Chemokine (C-C Motif) Ligand 17 (TARC), eotaxin 3, soluble vascular cell adhesion molecule 1 (sVCAM-1), thrombopoietin (TPO), fatty acid binding protein (FABP), IL-18, beta-2-microglobulin (B2M), serum amyloid A1 cluster (SAA), tenacin C, tumor necrosis factor receptor 1 (TNFR1), and optionally pancreatic polypeptide (PPY); (c) comparing the expression level of the one or more biomarkers in (b) with a statistical sample representative of the subject, thus determining whether the subject can be ruled out as having Parkinson's disease; and (d) determining that the subject is to be excluded from diagnostic testing for Parkinson's disease based on the comparing step. In certain embodiments, the method further comprises (e) avoiding, not commencing, or discontinuing a diagnostic test for Parkinson's disease, wherein the diagnostic test is selected from the group consisting of neurological examination, MRI, dopamine transporter (DAT) scan, brain ultrasounds, PET scan, detailed neuropsychological testing, and any combinations thereof. In certain embodiments, the method further comprises (f) avoiding, not commencing, or discontinuing a treatment for Parkinson's disease, wherein the treatment is selected from the group consisting of levodopa, a dopamine agonist, a glutamate agonist, an anticholinergic agent, a catechol-o-methyl transferase (COMT) inhibitor, a monoamine oxidase type B (MAO-B) inhibitor, a dopaminergic therapy, an N-methyl-D-aspartate (NMDA) antagonist, and any combinations thereof. In certain embodiments, the one or more biomarkers are selected from the group consisting of tenacin C, IL-6, 1309, IL-7, and FABP. In certain embodiments, the expression level of each biomarker in the group consisting of tenacin C, IL-6, 1309, and IL-7 is measured; and optionally the expression level of one or more biomarkers selected from the group consisting of FABP, TNFα, IL-5, CRP, IL-10, sICAM-1, Factor VII, 1309, A2M, TARC, eotaxin 3, sVCAM-1, TPO, IL-18, B2M, SAA, and PPY is measured. In certain embodiments, the expression level of the one or more biomarkers is measured using electrochemiluminescence. In certain embodiments, (i) when the expression level of the one or more biomarkers in (b) is statistically similar to the average expression level of the corresponding one or more biomarkers obtained from a group of individuals in the statistical sample who do not have Parkinson's disease, the subject is excluded from diagnostic testing for Parkinson's disease; or (ii) when the expression level of the one or more biomarkers in (b) is not statistically similar to the average expression level of the corresponding one or more biomarkers obtained from a group of individuals in the statistical sample who have been diagnosed with Parkinson's disease, the subject is excluded from diagnostic testing for Parkinson's disease.

In another aspect the present disclosure relates to a method for excluding a subject from recruitment into a clinical trial for an investigational Parkinson's disease medication, the method comprising: (a) obtaining a blood, plasma, or serum sample from the subject; (b) measuring in the blood, plasma, or serum sample the expression level of one or more biomarkers selected from the group consisting of: interleukin (IL)-7, tumor necrosis factor alpha (TNFα), IL-5, IL-6, C-reactive protein (CRP), IL-10, soluble intracellular adhesion molecule (sICAM-1), Factor VII, 1309, alpha-2-microglobulin (A2M), Chemokine (C-C Motif) Ligand 17 (TARC), eotaxin 3, soluble vascular cell adhesion molecule 1 (sVCAM-1), thrombopoietin (TPO), fatty acid binding protein (FABP), IL-18, beta-2-microglobulin (B2M), serum amyloid A1 cluster (SAA), tenacin C, tumor necrosis factor receptor 1 (TNFR1), and optionally pancreatic polypeptide (PPY); (c) comparing the expression level of the one or more biomarkers in (b) with a statistical sample representative of the subject; and (d) excluding the subject from recruitment into the clinical trial if the subject is ruled out of having Parkinson's disease from the comparison with the statistical sample. In certain embodiments, the method further comprises (e) avoiding, not commencing, or discontinuing a diagnostic test for Parkinson's disease, wherein the diagnostic test is selected from neurological examination, MRI, dopamine transporter (DAT) scan, brain ultrasound, PET scan, and detailed neuropsychological testing. In certain embodiments, the method further comprises (f) avoiding, not commencing, or discontinuing a treatment for Parkinson's disease, wherein the treatment is selected from levodopa, a dopamine agonist, a glutamate agonist, an anticholinergic agent, a catechol-o-methyl transferase (COMT) inhibitor, a monoamine oxidase type B (MAO-B) inhibitor, a dopaminergic therapy, an N-methyl-D-aspartate (NMDA) antagonist, and any combinations thereof. In certain embodiments, the one or more biomarkers are selected from the group consisting of tenacin C, IL-6, 1309, IL-7, and FABP. In certain embodiments, the expression level of each biomarker in the group consisting of tenacin C, IL-6, 1309, and IL-7 is measured; and optionally the expression level of one or more biomarkers selected from the group consisting of FABP, TNFα, IL-5, CRP, IL-10, sICAM-1, Factor VII, 1309, A2M, TARC, eotaxin 3, sVCAM-1, TPO, IL-18, B2M, SAA, and PPY is measured. In certain embodiments, the expression level of the one or more biomarkers is measured using electrochemiluminescence. In certain embodiments, (i) when the expression level of the one or more biomarkers in (b) is statistically similar to the average expression level of the corresponding one or more biomarkers obtained from a group of individuals in the statistical sample who do not have Parkinson's disease, the subject is excluded from recruitment into the clinical study; or (ii) the expression level of the one or more biomarkers in (b) is not statistically similar to the average expression level of the corresponding one or more biomarkers obtained from a group of individuals in the statistical sample who have been diagnosed with Parkinson's disease, the subject is excluded from recruitment into the clinical study.

In another aspect, the present disclosure relates to a method of for screening a subject to determine whether the subject is ruled out as having Parkinson's disease, the method comprising: (a) obtaining a blood, plasma, or serum sample from the subject; (b) measuring in the blood, plasma, or serum sample the expression level of one or more biomarkers selected from the group consisting of: interleukin (IL)-7, tumor necrosis factor alpha (TNFα), IL-5, IL-6, C-reactive protein (CRP), IL-10, soluble intracellular adhesion molecule (sICAM-1), Factor VII, 1309, alpha-2-microglobulin (A2M), Chemokine (C-C Motif) Ligand 17 (TARC), eotaxin 3, soluble vascular cell adhesion molecule 1 (sVCAM-1), thrombopoietin (TPO), fatty acid binding protein (FABP), IL-18, beta-2-microglobulin (B2M), serum amyloid A1 cluster (SAA), tenacin C, tumor necrosis factor receptor 1 (TNFR1), and optionally pancreatic polypeptide (PPY); (c) comparing the expression level of the one or more biomarkers in (b) with a statistical sample representative of the subject, thus determining whether the subject is ruled out as having Parkinson's disease; and (d) excluding the subjects who are ruled out as having Parkinson's disease from a diagnostic test for Parkinson's disease, a treatment of Parkinson's disease, or a combination thereof; or (e) administering a diagnostic test for Parkinson's disease, a treatment for Parkinson's disease, or a combination thereof to the subjects who are not ruled out as having Parkinson's disease. In certain embodiments, the diagnostic test is selected from the group consisting of neurological examination, MRI, dopamine transporter (DAT) scan, brain ultrasound, PET scan, detailed neuropsychological testing, and any combinations thereof. In certain embodiments, the treatment is selected from the group consisting of levodopa, a dopamine agonist, a glutamate agonist, an anticholinergic agent, a catechol-o-methyl transferase (COMT) inhibitor, a monoamine oxidase type B (MAO-B) inhibitor, a dopaminergic therapy, an N-methyl-D-aspartate (NMDA) antagonist, and any combinations thereof. In certain embodiments, the one or more biomarkers are selected from the group consisting of tenacin C, IL-6, 1309, IL-7, and FABP. In certain embodiments, the expression level of each biomarker in the group consisting of tenacin C, IL-6, I309, and IL-7 is measured; and optionally the expression level of one or more biomarkers selected from the group consisting of FABP, TNFα, IL-5, CRP, IL-10, sICAM-1, Factor VII, 1309, A2M, TARC, eotaxin 3, sVCAM-1, TPO, IL-18, B2M, SAA, and PPY is measured. In certain embodiments, the expression level of the one or more biomarkers is measured using electrochemiluminescence. In certain embodiments, (i) when the expression level of the one or more biomarkers in (b) is not statistically similar to the average expression level of the corresponding one or more biomarkers obtained from a group of individuals in the statistical sample who do not have Parkinson's disease, the subject is not ruled out as having Parkinson's disease; or (ii) when the expression level of the one or more biomarkers in (b) is statistically similar to the average expression level of the corresponding one or more biomarkers obtained from a group of individuals in the statistical sample who have been diagnosed with Parkinson's disease, the subject is not ruled out as having Parkinson's disease. In certain embodiments, the method further comprises (f) referring the subjects not ruled out as having Parkinson's disease to a specialist in Parkinson's disease.

In another aspect, the present disclosure relates to a method for excluding a subject from the need for diagnostic testing for Parkinson's disease, the method comprising: (a) obtaining a blood, plasma, or serum sample from the subject; (b) measuring in the blood, plasma, or serum sample the expression level of one or more biomarkers selected from the group consisting of: interleukin (IL)-7, tumor necrosis factor alpha (TNFα), IL-5, IL-6, C-reactive protein (CRP), IL-10, soluble intracellular adhesion molecule (sICAM-1), Factor VII, 1309, alpha-2-microglobulin (A2M), Chemokine (C-C Motif) Ligand 17 (TARC), eotaxin 3, soluble vascular cell adhesion molecule 1 (sVCAM-1), thrombopoietin (TPO), fatty acid binding protein (FABP), IL-18, beta-2-microglobulin (B2M), serum amyloid A1 cluster (SAA), tenacin C, TNRF1, and optionally pancreatic polypeptide (PPY); (c) comparing, using a computer, the expression level of the one or more biomarkers in (b) with a statistical sample representative of the subject, thus determining whether the subject can be ruled out as having Parkinson's disease; and (d) determining, using a computer, that the subject is to be excluded from diagnostic testing for Parkinson's disease based on the comparing step. In certain embodiments, the method further comprises (e) avoiding, not commencing, or discontinuing a diagnostic test for Parkinson's disease, wherein the diagnostic test is selected from the group consisting of neurological examination, MRI, dopamine transporter (DAT) scan, brain ultrasound, PET scan, detailed neuropsychological testing, and any combinations thereof. In certain embodiments, the method further comprises (f) avoiding, not commencing, or discontinuing a treatment for Parkinson's disease, wherein the treatment is selected from the group consisting of levodopa, a dopamine agonist, a glutamate agonist, an anticholinergic agent, a catechol-o-methyl transferase (COMT) inhibitor, a monoamine oxidase type B (MAO-B) inhibitor, a dopaminergic therapy, an N-methyl-D-aspartate (NMDA) antagonist, and any combinations thereof. In certain embodiments, the one or more biomarkers are selected from the group consisting of tenacin C, IL-6, 1309, IL-7, and FABP. In certain embodiments, the expression level of each biomarker in the group consisting of tenacin C, IL-6, 1309, and IL-7 is measured; and optionally the expression level of one or more biomarkers selected from the group consisting of FABP, TNFα, IL-5, CRP, IL-10, sICAM-1, Factor VII, 1309, A2M, TARC, eotaxin 3, sVCAM-1, TPO, IL-18, B2M, SAA, and PPY is measured. In certain embodiments, the expression level of the one or more biomarkers is measured using electrochemiluminescence. In certain embodiments, (i) when the expression level of the one or more biomarkers in (b) is determined by a computer to be statistically similar to the average expression level of the corresponding one or more biomarkers obtained from a group of individuals in the statistical sample who do not have Parkinson's disease, the subject is excluded from diagnostic testing for Parkinson's disease; or (ii) when the expression level of the one or more biomarkers in (b) is determined by a computer that is not statistically similar to the average expression level of the corresponding one or more biomarkers obtained from a group of individuals in the statistical sample who have been diagnosed with Parkinson's disease, the subject is excluded from diagnostic testing for Parkinson's disease.

### BRIEF DESCRIPTION OF THE DRAWINGS

The following detailed description of selected embodiments of the invention will be better understood when read in conjunction with the appended drawings. For the purpose of illustrating the invention, there are shown in the drawings illustrative embodiments. It should be understood, however, that the invention is not limited to the precise arrangements and instrumentalities of the embodiments shown in the drawings.

FIG. 1 shows the support vector machines (SVM)-based Parkinson's disease blood test (PDBT) accuracy and variable importance plot of the training set.

FIG. 2 shows the SVM-based PDBT accuracy and ROC curve of the test set.

### DESCRIPTION OF THE DISCLOSURE

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity but include the general class of which a specific example may be used for illustration. The terminology herein is used to describe specific embodiments of the invention, but their usage does not delimit the invention, except as outlined in the claims.

The differential diagnosis of neurodegenerative diseases is difficult, yet of critical importance for clinical treatment and management as well as for designing therapeutic and prevention trials. In order for patients to be referred to specialty clinics for diagnostic testing and treatment implementation, an appropriate referral is normally required from primary care providers. However, prior work demonstrates that the assessment and management of neurodegenerative diseases is poor in primary care settings with inappropriate medications frequently administered. Given that the average physician visit duration in an ambulatory setting for those age 65+ is approximately 18 minutes, primary care providers are in desperate need for rapid and cost-effective screens for ruling out neurological illness within their patients so appropriate referrals to a specialist can be made as warranted.

The availability of blood-based screening tools that can be implemented within primary care clinic settings has significant implications. From a clinical standpoint, while fewer than half of physicians surveyed believed screenings for neurodegenerative disease was important, the vast majority of the general public and caregivers believed such screenings were vitally important. Additionally, the average physician visit is less than 20 minutes for elderly patients in an ambulatory setting, severely limiting the time available for even brief neurological and cognitive assessments. Therefore, primary care providers are in desperate need of screening tools for ruling out patients from referral to a specialist for diagnostic testing of possible neurodegenerative disease. While a tremendous amount of work has been completed demonstrating the utility of diagnostic tests (MRI, fMRI, DTI, PET) for neurodegenerative diseases, they are cost- and time-prohibitive for ruling out disease in a primary care setting.

Within primary care settings, the role of screening tests is to rule out patients who do not require additional medical procedures or diagnostic follow-up, thereby resulting in stress reduction and cost containment. While application of specialty clinic-based diagnostic tests as screens for ruling out disease in primary care settings seems straight forward, this is not the case and no prior procedures will work within primary care settings as demonstrated below. The ability to implement blood-based screens to rule out disease in a primary care setting is critical, yet very complicated due to substantially lower base rates of disease presence as compared to specialty clinics and this lower base rate has a tremendous impact on the predictive accuracy of test results.

Some recent progress has been made in the development of biomarker-based tests to detect individuals with neurodegenerative disease and distinguishing a single disease from other neurodegenerative diseases, including Alzheimer's disease (AD), PD, Dementia with Lewy Bodies (DLB), and AD among adults with Down Syndrome (DS-AD). Over the last several decades, the search for biomarkers that have diagnostic and prognostic utility in neurodegenerative diseases has grown exponentially, with the majority of work focusing on neuroimaging and cerebrospinal (CSF) methodologies. In fact, the dopamine transporter single photon emission CT [DaT-SPECT] has been approved as a test for diagnosing PD. In addition to blood-based biomarkers, research suggests that CSF markers may also hold utility in the differential diagnosis of neurodegenerative diseases. While advanced neuroimaging, blood-based markers, and CSF methods have tremendous potential as diagnostic tests for PD, invasiveness, accessibility, cost barriers, and technical validity have so far limited the utility of such methods to diagnostic testing applications. To date, these methods have not been found suitable as screens for ruling out disease in a primary care setting.

The present disclosure relates, in part, to a method for excluding a subject from the need for diagnostic testing for Parkinson's disease. In some embodiments, the present disclosure provides a method for ruling out PD in a subject. In some embodiments, the disclosed method can be used in a primary care setting to rule out PD in a subject. In some embodiments, the method comprises measuring the expression level of one or more biomarkers selected from the group consisting of IL-7, TNFα, IL-5, IL-6, CRP, IL-10, sICAM-1, Factor VII, 1309, A2M, TARC, eotaxin 3, sVCAM-1, TPO, FABP3, IL18, B2M, SAA, tenacin C, TNFR1, and optionally PPY.

In some embodiments, the one or more biomarkers comprises IL-7. In some embodiments, the one or more biomarkers comprises TNFα. In some embodiments, the one or more biomarkers comprises IL-5. In some embodiments, the one or more biomarkers comprises IL-6. In some embodiments, the one or more biomarkers comprises CRP. In some embodiments, the one or more biomarkers comprises IL-10. In some embodiments, the one or more biomarkers comprises sICAM-1. In some embodiments, the one or more biomarkers comprises Factor VII. In some embodiments, the one or more biomarkers comprises 1309. In some embodiments, the one or more biomarkers comprises A2M. In some embodiments, the one or more biomarkers comprises TARC. In some embodiments, the one or more biomarkers comprises eotaxin 3. In some embodiments, the one or more biomarkers comprises sVCAM-1. In some embodiments, the one or more biomarkers comprises TPO. In some embodiments, the one or more biomarkers comprises FABP3. In some embodiments, the one or more biomarkers comprises IL18. In some embodiments, the one or more biomarkers comprises B2M. In some embodiments, the one or more biomarkers comprises SAA. In some embodiments, the one or more biomarkers comprises tenacin C. In some embodiments, the one or more biomarkers comprises TNFR1. In some embodiments, the one or more biomarkers comprises PPY.

In some embodiments, the one or more biomarkers comprise tenacin C. In some embodiments, the one or more biomarkers comprise tenacin C and IL-6. In some embodiments, the one or more biomarkers comprise tenacin C, IL-6, and 1309. In some embodiments, the one or more biomarkers comprise tenacin C, IL-6, 1309, and IL-7. In some embodiments, the one or more biomarkers comprise tenacin C, IL-6, 1309, IL-7, and FABP3. In some embodiments, the one or more biomarkers comprise tenacin C, IL-6, 1309, IL-7, FABP3, and TARC. In some embodiments, the one or more biomarkers comprise tenacin C, IL-6, 1309, IL-7, FABP3, TARC, and TNFα. In some embodiments, the one or more biomarkers comprise tenacin C, IL-6, 1309, IL-7, FABP3, TARC, TNFα, and A2M. In some embodiments, the one or more biomarkers comprise tenacin C, IL-6, 1309, IL-7, FABP3, TARC, TNFα, A2M, and TPO. In some embodiments, the one or more biomarkers comprise tenacin C, IL-6, 1309, IL-7, FABP3, TARC, TNFα, A2M, TPO, and B2M. In some embodiments, the one or more biomarkers comprise tenacin C, IL-6, 1309, IL-7, FABP3, TARC, TNFα, A2M, TPO, B2M, and IL5. In some embodiments, the one or more biomarkers comprise tenacin C, IL-6, 1309, IL-7, FABP3, TARC, TNFα, A2M, TPO, B2M, IL5, and Factor VII. In some embodiments, the one or more biomarkers comprise tenacin C, IL-6, 1309, IL-7, FABP3, TARC, TNFα, A2M, TPO, B2M, IL5, Factor VII, and IL10. In some embodiments, the one or more biomarkers comprise tenacin C, IL-6, 1309, IL-7, FABP3, TARC, TNFα, A2M, TPO, B2M, IL5, Factor VII, IL10, sVCAM-1. In some embodiments, the one or more biomarkers comprise tenacin C, IL-6, 1309, IL-7, FABP3, TARC, TNFα, A2M, TPO, B2M, IL5, Factor VII, IL10, sVCAM-1, and eotaxin 3. In some embodiments, the one or more biomarkers comprise tenacin C, IL-6, 1309, IL-7, FABP3, TARC, TNFα, A2M, TPO, B2M, IL5, Factor VII, IL10, sVCAM-1, eotaxin 3, and SAA. In some embodiments, the one or more biomarkers comprise tenacin C, IL-6, 1309, IL-7, FABP3, TARC, TNFα, A2M, TPO, B2M, IL5, Factor VII, IL10, sVCAM-1, eotaxin 3, SAA, and IL18. In some embodiments, the one or more biomarkers comprise tenacin C, IL-6, 1309, IL-7, FABP3, TARC, TNFα, A2M, TPO, B2M, IL5, Factor VII, IL10, sVCAM-1, eotaxin 3, SAA, IL18, and sICAM-1. In some embodiments, the one or more biomarkers comprise tenacin C, IL-6, 1309, IL-7, FABP3, TARC, TNFα, A2M, TPO, B2M, IL5, Factor VII, IL10, sVCAM-1, eotaxin 3, SAA, IL18, sICAM-1, and CRP.

In certain embodiments, the expression level of the selected biomarkers are measured in a blood, plasma, or serum sample obtained from the subject. In some embodiments, the expression level of the one or more biomarkers is compared to the expression level of the corresponding one or more biomarkers in a statistical sample representative of the subject, wherein the comparison is used to determine if the subject warrants diagnostic screening for Parkinson's disease or if the subject can be excluded from diagnostic testing for Parkinson's disease. In some embodiments, the method for excluding a subject from the need for diagnostic testing for Parkinson's disease can be performed in a primary care setting.

In another aspect, the present disclosure relates to a method for excluding a subject from recruitment into a clinical trial for an investigational Parkinson's disease medication. In some embodiments, the method comprises measuring the expression level of one or more biomarkers selected from the group consisting of IL-7, TNFα, IL-5, IL-6, CRP, IL-10, sICAM-1, Factor VII, 1309, A2M, TARC, eotaxin 3, sVCAM-1, TPO, FABP3, IL18, B2M, SAA, tenacin C, TNRF1, and optionally PPY.

In some embodiments, the one or more biomarkers comprises IL-7. In some embodiments, the one or more biomarkers comprises TNFα. In some embodiments, the one or more biomarkers comprises IL-5. In some embodiments, the one or more biomarkers comprises IL-6. In some embodiments, the one or more biomarkers comprises CRP. In some embodiments, the one or more biomarkers comprises IL-10. In some embodiments, the one or more biomarkers comprises sICAM-1. In some embodiments, the one or more biomarkers comprises Factor VII. In some embodiments, the one or more biomarkers comprises 1309. In some embodiments, the one or more biomarkers comprises A2M. In some embodiments, the one or more biomarkers comprises TARC. In some embodiments, the one or more biomarkers comprises eotaxin 3. In some embodiments, the one or more biomarkers comprises sVCAM-1. In some embodiments, the one or more biomarkers comprises TPO. In some embodiments, the one or more biomarkers comprises FABP3. In some embodiments, the one or more biomarkers comprises IL18. In some embodiments, the one or more biomarkers comprises B2M. In some embodiments, the one or more biomarkers comprises SAA. In some embodiments, the one or more biomarkers comprises tenacin C. In some embodiments, the one or more biomarkers comprises TNFR1. In some embodiments, the one or more biomarkers comprises PPY

In some embodiments, the one or more biomarkers comprise tenacin C. In some embodiments, the one or more biomarkers comprise tenacin C and IL-6. In some embodiments, the one or more biomarkers comprise tenacin C, IL-6, and 1309. In some embodiments, the one or more biomarkers comprise tenacin C, IL-6, 1309, and IL-7. In some embodiments, the one or more biomarkers comprise tenacin C, IL-6, 1309, IL-7, and FABP3. In some embodiments, the one or more biomarkers comprise tenacin C, IL-6, 1309, IL-7, FABP3, and TARC. In some embodiments, the one or more biomarkers comprise tenacin C, IL-6, 1309, IL-7, FABP3, TARC, and TNFα. In some embodiments, the one or more biomarkers comprise tenacin C, IL-6, 1309, IL-7, FABP3, TARC, TNFα, and A2M. In some embodiments, the one or more biomarkers comprise tenacin C, IL-6, 1309, IL-7, FABP3, TARC, TNFα, A2M, and TPO. In some embodiments, the one or more biomarkers comprise tenacin C, IL-6, 1309, IL-7, FABP3, TARC, TNFα, A2M, TPO, and B2M. In some embodiments, the one or more biomarkers comprise tenacin C, IL-6, 1309, IL-7, FABP3, TARC, TNFα, A2M, TPO, B2M, and IL5. In some embodiments, the one or more biomarkers comprise tenacin C, IL-6, 1309, IL-7, FABP3, TARC, TNFα, A2M, TPO, B2M, IL5, and Factor VII. In some embodiments, the one or more biomarkers comprise tenacin C, IL-6, 1309, IL-7, FABP3, TARC, TNFα, A2M, TPO, B2M, IL5, Factor VII, and IL10. In some embodiments, the one or more biomarkers comprise tenacin C, IL-6, 1309, IL-7, FABP3, TARC, TNFα, A2M, TPO, B2M, IL5, Factor VII, IL10, sVCAM-1. In some embodiments, the one or more biomarkers comprise tenacin C, IL-6, 1309, IL-7, FABP3, TARC, TNFα, A2M, TPO, B2M, IL5, Factor VII, IL10, sVCAM-1, and eotaxin 3. In some embodiments, the one or more biomarkers comprise tenacin C, IL-6, 1309, IL-7, FABP3, TARC, TNFα, A2M, TPO, B2M, IL5, Factor VII, IL10, sVCAM-1, eotaxin 3, and SAA. In some embodiments, the one or more biomarkers comprise tenacin C, IL-6, 1309, IL-7, FABP3, TARC, TNFα, A2M, TPO, B2M, IL5, Factor VII, IL10, sVCAM-1, eotaxin 3, SAA, and IL18. In some embodiments, the one or more biomarkers comprise tenacin C, IL-6, 1309, IL-7, FABP3, TARC, TNFα, A2M, TPO, B2M, IL5, Factor VII, IL10, sVCAM-1, eotaxin 3, SAA, IL18, and sICAM-1. In some embodiments, the one or more biomarkers comprise tenacin C, IL-6, 1309, IL-7, FABP3, TARC, TNFα, A2M, TPO, B2M, IL5, Factor VII, IL10, sVCAM-1, eotaxin 3, SAA, IL18, sICAM-1, and CRP.

In some embodiments, the expression level of the one or more biomarkers is compared to the expression level of the corresponding one or more biomarkers in a statistical sample representative of the subject, wherein the comparison is used to determine if the subject can be ruled out as having Parkinson's disease and therefore should be excluded from the clinical trial.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element. “About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein, the phrase “primary care clinic”, “primary care setting”, “primary care provider” are used interchangeably to refer to the principal point of contact/consultation for patients within a health care system and coordinates with specialists that the patient may need.

As used herein, the phrase “specialist” refers to a medical practice or practitioner that specializes in a particular disease, such as neurology, psychiatry or even more specifically movement disorders or memory disorders.

As used herein, the phrase “screening out” or “ruling out” refers to a blood, serum, or plasma test that rules out Parkinson's Disease (PD). Conversely, those subjects that are not screened- or ruled-out as having PD would be recommended for actual diagnostic testing for PD. Thus, the present disclosure is a screening test and not a diagnostic test. The present disclosure further provides the first screening blood test that can be used in a primary care setting for screening out PD.

As used herein, the following abbreviations are used and can include mammalian version of these genes or gene products but in certain embodiments the genes or gene products are human genes or gene products: IL7-interleukin-7, TNFα-tumor necrosis factor alpha, IL5-interleukin-5, IL6-interleukin-6, CRP-C-reactive protein, IL10-interleukin-10, TNC-Tenascin C, ICAM1-intracellular adhesion molecule 1, FVII-factor VII, 1309-chemokine (C-C motif) ligand 1, TNFR1-tumor necrosis factor receptor 1, A2M-alpha-2-microglobulin, TARC-Chemokine (C-C Motif) Ligand 17, eotaxin3, VCAM1-Vascular Cell Adhesion Molecule 1, TPO-Thrombopoietin, FABP3-fatty acid binding protein 3, IL18-interleukin-18, B2M-beta-2-microglobulin, SAA-serum amyloid A1 cluster, PPY-pancreatic polypeptide, DJ1-Parkinson Protein 7, α-syn-α-synuclein.

As used herein, FABP and FABP3 are interchangeable.

As used herein, intracellular adhesion molecule 1 (ICAM1, ICAM-1) is interchangeable with soluble intracellular adhesion molecule 1 (sICAMI, sICAM-1).

As used herein, vascular cell adhesion molecule 1 (VCAM1, VCAM-1) is interchangeable with soluble vascular cell adhesion molecule 1 (sVCAM1, sVCAM-1).

As used herein, the phrase “neurological disease” refers to a disease or disorder of the central nervous system and many include, e.g., neurodegenerative disorders such as Alzheimer's disease (AD), Parkinson's disease (PD), mild cognitive impairment (MCI), Frontotemporal dementia (FTD), Dementia with Lewy Bodies (DLB), Down's syndrome (DS), and dementia and neurological diseases including multiple sclerosis and neuropathies.

As used herein, the terms “Parkinson's disease patient”, and “individual diagnosed with Parkinson's disease” all refer to an individual who has been diagnosed with PD or has been given a diagnosis of Parkinson's disease.

As used herein, the phrase “Parkinson's disease biomarker” refers to a biomarker that is used to screen/rule out Parkinson's Disease (PD).

As used herein, the term “Parkinson's disease biomarker protein” refers to any protein biomarker or substance that is functionally at the level of a protein biomarker measured to screen/rule out Parkinson's Disease (PD).

As used herein, a “blood sample” refers to a biological sample derived from blood, preferably peripheral (or circulating) blood. A blood sample may be, e.g., whole blood, serum or plasma. In certain embodiments, serum is preferred as the source for the biomarkers as the samples are readily available and often obtained for other sampling, is stable, and requires less processing, thus making it ideal for locations with little to refrigeration or electricity, is easily transportable, and is commonly handled by medical support staff.

As used herein, a “normal” individual or a sample from a “normal” individual refers to quantitative data, qualitative data, or both from an individual who has or would be assessed by a physician as not having a disease, e.g., a neurological disease. Often, a “normal” individual is also age-matched within a range of 1, 2, 3, 4, 5, 6, 7, 8, 9 or 10 years with the sample of the individual to be assessed.

As used herein, the term “treatment” refers to the alleviation, amelioration, and/or stabilization of symptoms, as well as delay in progression of symptoms of a particular disorder. For example, “treatment” of PD includes any one or more of: (1) elimination of one or more symptoms of PD, (2) reduction of one or more symptoms of PD, (3) stabilization of the symptoms of PD (e.g., failure to progress to more advanced stages of PD), and (4) delay in onset of one or more symptoms of PD; and (5) delay in progression (i.e., worsening) of one or more symptoms of PD.

As used herein, the phrase “neurocognitive evaluations” is used to describe one or more tests known to the skilled artisan for measuring cognitive status or impairment and can include but is not limited to: a 4-point clock drawing test, an verbal fluency test, trail making test, list learning test, and the like. The skilled artisan will recognize and know how these tests can be modified, how new tests that measure similar cognitive function can be developed and implemented for use with the present invention.

### DESCRIPTION

### Methods

In one aspect, the present disclosure provides a method for excluding a subject from the need for diagnostic testing for Parkinson's disease. In certain embodiments, the method comprises: (a) obtaining a blood, plasma or, serum sample from the subject; (b) measuring in the blood, plasma, or serum sample the expression level of one or more biomarkers selected from the group consisting of: IL-7, TNFα, IL-5, IL-6, CRP, IL-10, sICAM-1, Factor VII, 1309, A2M, TARC, eotaxin 3, sVCAM-1, TPO, FABP3, IL18, B2M, SAA, tenacin C, TNFR1, and optionally PPY; (c) comparing the expression level of the one or more biomarkers in (b) with a statistical sample representative of the subject, thus determining whether the subject can be ruled out as having Parkinson's disease; and (d) determining that the comparing step indicates that the subject is excluded from diagnostic testing for Parkinson's disease.

In some embodiments, the method further comprises the step of avoiding, not commencing, or discontinuing diagnostic tests for Parkinson's disease, wherein the diagnostic tests are selected from MRI, dopamine transporter (DAT) scan, brain ultrasound, PET scan, and detailed neuropsychological testing. In some embodiments, the method further comprises the step of avoiding, not commencing, or discontinuing treatments for Parkinson's disease, wherein the treatment is selected from levodopa, a dopamine agonist, a glutamate agonist, an anticholinergic agent, a catechol-o-methyl transferase (COMT) inhibitor, a monoamine oxidase type B (MAO-B) inhibitor, a dopaminergic therapy, an N-methyl-D-aspartate (NMDA) antagonist, and any combinations thereof.

In certain embodiments the subject is an elderly subject. In other embodiments, the subject is a middle aged subject. In some embodiments, the subject has concerns of motor changes (reported by self, physician, or other) or a family history of PD. In certain embodiments, the middle aged subject is 30 years of age or older. In certain embodiments, the subject is a human subject.

In certain embodiments, the sample is a blood sample. In some embodiments, the blood, plasma, or serum sample is obtained from the subject in a primary care setting.

In some embodiments, the statistical sample comprises a group of individuals 30 years of age and older who do not have a neurological disease or disorder. In other embodiments, the statistical sample comprises a group of individuals 30 years of age and older who have been diagnosed with a neurological disease or disorder. In some embodiments, the statistical sample comprises a group of individuals 30 years of age and older who have been diagnosed with PD. In some embodiments, the statistical sample comprises the measurements of the expression level for one or more biomarkers selected from IL-7, TNFα, IL-5, IL-6, CRP, IL-10, sICAM-1, Factor VII, 1309, A2M, TARC, eotaxin 3, sVCAM-1, TPO, FABP3, IL18, B2M, SAA, tenacin C, TNRF1, and optionally PPY from each individual in the statistical sample.

In certain embodiments, a statistically similar expression level of the one or more biomarkers in the subject's sample when compared to the corresponding biomarkers from individuals in the statistical sample who do not have a neurological disease or disorder indicates that diagnostic testing of the subject for PD is not needed. In another embodiment, a statistically similar expression level of the one or more biomarkers in the subject's sample when compared to the corresponding biomarkers of individuals in the statistical sample diagnosed with PD indicates that diagnostic testing of the subject for PD is needed. In some embodiments, the expression level of the one or more biomarkers measured in the subject's sample is compared to the average expression level of the corresponding biomarkers in the statistical sample for each group of individuals in the statistical sample.

In certain embodiments wherein the expression level of the one or more measured biomarkers indicates that diagnostic testing of the subject for PD is needed, the subject is referred to a PD specialist for diagnostic testing. In other embodiments wherein the expression level of the one or more measured biomarkers indicates rules out the subject as having PD, the subject is not referred to a PD specialist for diagnostic testing. Therefore, in some embodiments, the disclosed method prevents the subject from undergoing invasive and expensive tests that a specialist may prescribe to determine if the subject has PD.

In some embodiments, the step of measuring the expression level of the one or more biomarkers in the blood, plasma, or serum sample comprises measuring the expression level of each of tenacin C; tenacin C and IL-6; tenacin C, IL-6, and 1309; tenacin C, IL-6, 1309, and IL-7; tenacin C, IL-6, 1309, IL-7, and FABP; tenacin C, IL-6, 1309, IL-7, FABP3, and TARC; tenacin C, IL-6, 1309, IL-7, FABP3, TARC, and TNFα; tenacin C, IL-6, 1309, IL-7, FABP3, TARC, TNFα, and A2M; of tenacin C, IL-6, 1309, IL-7, FABP3, TARC, TNFα, A2M, and TPO; tenacin C, IL-6, 1309, IL-7, FABP3, TARC, TNFα, and A2M; tenacin C, IL-6, 1309, IL-7, FABP3, TARC, TNFα, A2M, TPO, and B2M; tenacin C, IL-6, 1309, IL-7, FABP3, TARC, TNFα, A2M, TPO, B2M, and IL-5; tenacin C, IL-6, 1309, IL-7, FABP3, TARC, TNFα, A2M, TPO, B2M, IL-5, and Factor VII; tenacin C, IL-6, 1309, IL-7, FABP3, TARC, TNFα, A2M, TPO, B2M, IL-5, Factor VII, and IL-10; tenacin C, IL-6, 1309, IL-7, FABP3, TARC, TNFα, A2M, TPO, B2M, IL-5, Factor VII, IL-10, and sVCAM-1; tenacin C, IL-6, 1309, IL-7, FABP3, TARC, TNFα, A2M, TPO, B2M, IL-5, Factor VII, IL-10, sVCAM-1, and eotaxin 3; tenacin C, IL-6, 1309, IL-7, FABP3, TARC, TNFα, A2M, TPO, B2M, IL-5, Factor VII, IL-10, sVCAM-1, eotaxin 3, and SAA; tenacin C, IL-6, 1309, IL-7, FABP3, TARC, TNFα, A2M, TPO, B2M, IL-5, Factor VII, IL-10, sVCAM-1, and eotaxin 3; tenacin C, IL-6, 1309, IL-7, FABP3, TARC, TNFα, A2M, TPO, B2M, IL-5, Factor VII, IL-10, sVCAM-1, eotaxin 3, SAA, and IL-18; tenacin C, IL-6, 1309, IL-7, FABP3, TARC, TNFα, A2M, TPO, B2M, IL-5, Factor VII, IL-10, sVCAM-1, and eotaxin 3; tenacin C, IL-6, 1309, IL-7, FABP3, TARC, TNFα, A2M, TPO, B2M, IL-5, Factor VII, IL-10, sVCAM-1, eotaxin 3, SAA, IL-18, and sICAM-1; tenacin C, IL-6, 1309, IL-7, FABP3, TARC, TNFα, A2M, TPO, B2M, IL-5, Factor VII, IL-10, sVCAM-1, eotaxin 3, SAA, IL-18, sICAM-1, and CRP. In some embodiments, the expression level of each of the biomarkers measured above is compared to the expression level of each of the corresponding biomarkers in the statistical sample to determine if the subject is excluded from further testing for Parkinson's disease.

In certain embodiments, the expression level of one or more of tenacin C, IL-6, 1309, IL-7, FABP3, TARC, TNFα, A2M, TPO, and B2M is measured in the blood, plasma, or serum sample. In certain embodiments, the expression level of each of tenacin C, IL-6, 1309, IL-7, FABP3, TARC, TNFα, A2M, TPO, and B2M is measured in the blood, plasma, or serum sample. In certain embodiments, the expression level of each of tenacin C, IL-6, 1309, and IL-7, and optionally FABP3, is measured in the blood, plasma, or serum sample.

In certain embodiments, the biomarker measurements are obtained by a method selected from the group consisting of an immunoassay, an enzymatic activity assay, fluorescence detection, chemiluminescence detection, electrochemiluminescence detection and patterned array, reverse transcriptase-polymerase chain reaction, antibody binding, fluorescence activated sorting, detectable bead sorting, antibody array, microarray, enzymatic array, receptor binding array, allele specific primer extension, target specific primer extension, solid-phase binding array, liquid phase binding array, fluorescent resonance transfer, and radioactive labeling. In some embodiments, the biomarker measurements are obtained by electrochemiluminescence detection.

In another aspect, the present disclosure relates to a method for excluding a subject from recruitment into a clinical trial for an investigational Parkinson's disease medication, the method comprising: (a) obtaining a blood, plasma, or serum sample from the subject; (b) measuring in the blood, plasma, or serum sample the expression level of one or more biomarkers selected from the group consisting of: IL-7, TNFα, IL-5, IL-6, CRP, IL-10, sICAM-1, Factor VII, 1309, A2M, TARC, eotaxin 3, sVCAM-1, TPO, FABP3, IL18, B2M, SAA, tenacin C, TNRF1, and optionally PPY; (c) comparing the expression level of the one or more biomarkers in (b) with a statistical sample representative of the subject; and (d) excluding the subject from recruitment into the clinical trial if the subject is ruled out of having Parkinson's disease from the comparison with the statistical sample.

In some embodiments, the method further comprises step (e) avoiding, not commencing, or discontinuing a diagnostic test for Parkinson's disease, wherein the diagnostic tests are selected from neurological examination, MRI, dopamine transporter (DAT) scan, brain ultrasound, PET scan, and detailed neuropsychological testing.

In some embodiments, the method further comprises step (f) avoiding, not commencing, or discontinuing a treatment for Parkinson's disease, wherein the treatment is selected from levodopa, a dopamine agonist, a glutamate agonist, an anticholinergic agent, a catechol-o-methyl transferase (COMT) inhibitor, a monoamine oxidase type B (MAO-B) inhibitor, a dopaminergic therapy, an N-methyl-D-aspartate (NMDA) antagonist, and any combinations thereof.

In some embodiments, step (d) is replaced with a step of excluding the subject from additional testing for recruitment into the clinical trial if the subject is ruled out of having Parkinson's disease from the comparison with the statistical sample. In some embodiments, the step of excluding the subject from additional testing for recruitment into the clinical trial if the subject is ruled out of having Parkinson's disease from the comparison with the statistical sample is followed by the step of excluding the subject from recruitment into the clinical trial if the subject is ruled out of having Parkinson's disease from the comparison with the statistical sample.

In certain embodiments the subject is an elderly subject. In other embodiments, the subject is a middle aged subject. In some embodiments, the subject has concerns of motor changes (reported by self, physician, or other) or a family history of PD. In certain embodiments, the middle aged subject is 30 years of age or older. In certain embodiments, the subject is a human subject.

In certain embodiments, the sample is a blood sample. In some embodiments, the blood, plasma, or serum sample is obtained from the subject in a primary care setting.

In some embodiments, the statistical sample comprises a group of individuals 30 years of age or older who do not have a neurological disease or disorder. In other embodiments, the statistical sample comprises a group of individuals 30 years of age or older who have been diagnosed with a neurological disease or disorder. In some embodiments, the statistical sample comprises a group of individuals 30 years of age or older who have been diagnosed with PD. In some embodiments, the statistical sample comprises the measurements of the expression level for one or more biomarkers selected from IL-7, TNFα, IL-5, IL-6, CRP, IL-10, sICAM-1, Factor VII, 1309, A2M, TARC, eotaxin 3, sVCAM-1, TPO, FABP3, IL18, B2M, SAA, tenacin C, TNRF1, and optionally PPY from each individual in the statistical sample.

In certain embodiments, a statistically similar expression level of the one or more biomarkers in the subject's sample when compared to the expression level of the corresponding biomarkers from individuals in the statistical sample who do not have a neurological disease or disorder indicates that the subject is ruled out as having PD and that the subject should be excluded from the clinical trial. In another embodiment, a statistically similar expression level of the one or more biomarkers in the subject's sample when compared to the corresponding biomarkers of individuals in the statistical sample diagnosed with PD indicates that the subject has not been ruled out as having PD and that the subject should not be excluded from the clinical trial. In some embodiments, the expression level of the one or more biomarkers measured in the subject's sample is compared to the average expression level of the corresponding biomarkers in the statistical sample for each group of individuals in the statistical sample.

In some embodiments, the subjects who have not been ruled out as having PD are referred to a specialist for diagnostic tests for PD before being recruited into the clinical trial. Exemplary diagnostic tests for PD are described elsewhere herein. In some embodiments, subjects diagnosed by the specialist as having PD are recruited into the clinical trial while subjects who the specialist determines do not have PD are not recruited into the clinical trial.

In some embodiments, the step of measuring the expression level of the one or more biomarkers in the blood, plasma, or serum sample comprises measuring the expression level of each of tenacin C; tenacin C and IL-6; tenacin C, IL-6, and 1309; tenacin C, IL-6, 1309, and IL-7; tenacin C, IL-6, 1309, IL-7, and FABP; tenacin C, IL-6, 1309, IL-7, FABP3, and TARC; tenacin C, IL-6, 1309, IL-7, FABP3, TARC, and TNFα; tenacin C, IL-6, 1309, IL-7, FABP3, TARC, TNFα, and A2M; of tenacin C, IL-6, 1309, IL-7, FABP3, TARC, TNFα, A2M, and TPO; tenacin C, IL-6, 1309, IL-7, FABP3, TARC, TNFα, and A2M; tenacin C, IL-6, 1309, IL-7, FABP3, TARC, TNFα, A2M, TPO, and B2M; tenacin C, IL-6, 1309, IL-7, FABP3, TARC, TNFα, A2M, TPO, B2M, and IL-5; tenacin C, IL-6, 1309, IL-7, FABP3, TARC, TNFα, A2M, TPO, B2M, IL-5, and Factor VII; tenacin C, IL-6, 1309, IL-7, FABP3, TARC, TNFα, A2M, TPO, B2M, IL-5, Factor VII, and IL-10; tenacin C, IL-6, 1309, IL-7, FABP3, TARC, TNFα, A2M, TPO, B2M, IL-5, Factor VII, IL-10, and sVCAM-1; tenacin C, IL-6, 1309, IL-7, FABP3, TARC, TNFα, A2M, TPO, B2M, IL-5, Factor VII, IL-10, sVCAM-1, and eotaxin 3; tenacin C, IL-6, 1309, IL-7, FABP3, TARC, TNFα, A2M, TPO, B2M, IL-5, Factor VII, IL-10, sVCAM-1, eotaxin 3, and SAA; tenacin C, IL-6, 1309, IL-7, FABP3, TARC, TNFα, A2M, TPO, B2M, IL-5, Factor VII, IL-10, sVCAM-1, and eotaxin 3; tenacin C, IL-6, 1309, IL-7, FABP3, TARC, TNFα, A2M, TPO, B2M, IL-5, Factor VII, IL-10, sVCAM-1, eotaxin 3, SAA, and IL-18; tenacin C, IL-6, 1309, IL-7, FABP3, TARC, TNFα, A2M, TPO, B2M, IL-5, Factor VII, IL-10, sVCAM-1, and eotaxin 3; tenacin C, IL-6, 1309, IL-7, FABP3, TARC, TNFα, A2M, TPO, B2M, IL-5, Factor VII, IL-10, sVCAM-1, eotaxin 3, SAA, IL-18, and sICAM-1; tenacin C, IL-6, 1309, IL-7, FABP3, TARC, TNFα, A2M, TPO, B2M, IL-5, Factor VII, IL-10, sVCAM-1, eotaxin 3, SAA, IL-18, sICAM-1, and CRP. In some embodiments, the expression level of each of the biomarkers measured above is compared to the expression level of each of the corresponding biomarkers in the statistical sample to determine if subject has been ruled out as having PD and should be excluded from the clinical trial.

In certain embodiments, the expression level of one or more of tenacin C, IL-6, 1309, IL-7, FABP3, TARC, TNFα, A2M, TPO, and B2M is measured in the blood or serum sample. In certain embodiments, the expression level of each of tenacin C, IL-6, 1309, IL-7, FABP3, TARC, TNFα, A2M, TPO, and B2M is measured in the blood, plasma, or serum sample. In certain embodiments, the expression level of each of tenacin C, IL-6, 1309, and IL-7, and optionally FABP3, is measured in the blood, plasma, or serum sample.

In certain embodiments, the biomarker measurements are obtained by a method selected from the group consisting of an immunoassay, an enzymatic activity assay, fluorescence detection, chemiluminescence detection, electrochemiluminescence detection and patterned array, reverse transcriptase-polymerase chain reaction, antibody binding, fluorescence activated sorting, detectable bead sorting, antibody array, microarray, enzymatic array, receptor binding array, allele specific primer extension, target specific primer extension, solid-phase binding array, liquid phase binding array, fluorescent resonance transfer, and radioactive labeling. In some embodiments, the biomarker measurements are obtained by electrochemiluminescence detection.

In yet another aspect, the present disclosure relates to a method of screening a subject to determine whether the subject is ruled out as having Parkinson's disease, the method comprising: (a) obtaining a blood, plasma, or serum sample from the subject; (b) measuring in the blood or serum sample the expression level of one or more biomarkers selected from the group consisting of: interleukin (IL)-7, tumor necrosis factor alpha (TNFα), IL-5, IL-6, C-reactive protein (CRP), IL-10, soluble intracellular adhesion molecule (sICAM-1), Factor VII, 1309, alpha-2-microglobulin (A2M), Chemokine (C-C Motif) Ligand 17 (TARC), eotaxin 3, soluble vascular cell adhesion molecule 1 (sVCAM-1), thrombopoietin (TPO), fatty acid binding protein (FABP), IL-18, beta-2-microglobulin (B2M), serum amyloid A1 cluster (SAA), tenacin C, TNRF1, and optionally pancreatic polypeptide (PPY); (c) comparing expression level of the one or more biomarkers in (b) with a statistical sample representative of the subject, thus determining whether the subject is ruled out or is not ruled out as having Parkinson's disease; and (d) excluding the subjects who are ruled out as having Parkinson's disease from diagnostic testing for Parkinson's disease, a treatment of Parkinson's disease, or a combination thereof; or (e) administering a diagnostic test for Parkinson's disease, a treatment for Parkinson's disease, or a combination thereof to the subjects who are not ruled out as having Parkinson's disease.

In certain embodiments the subject is an elderly subject. In other embodiments, the subject is a middle aged subject. In some embodiments, the subject has concerns of motor changes (reported by self, physician, or other) or a family history of PD. In certain embodiments, the middle aged subject is 30 years of age or older. In certain embodiments, the subject is a human subject.

In certain embodiments, the sample is a blood sample. In some embodiments, the blood, plasma, or serum sample is obtained from the subject in a primary care setting.

In some embodiments, the statistical sample comprises a group of individuals 30 years of age and older who do not have a neurological disease or disorder. In other embodiments, the statistical sample comprises a group of individuals 30 years of age and older who have been diagnosed with a neurological disease or disorder. In some embodiments, the statistical sample comprises a group of individuals 30 years of age and older who have been diagnosed with PDf. In some embodiments, the statistical sample comprises the measurements of the expression level for one or more biomarkers selected from IL-7, TNFα, IL-5, IL-6, CRP, IL-10, sICAM-1, Factor VII, 1309, A2M, TARC, eotaxin 3, sVCAM-1, TPO, FABP3, IL18, B2M, SAA, tenacin C, TNRF1, and optionally PPY from each individual in the statistical sample.

In certain embodiments, when the expression level of the one or more biomarkers in the subject's blood, plasma, or serum sample is not statistically similar to the expression level of the corresponding one or more biomarkers obtained from a group of individuals in the statistical sample who do not have Parkinson's disease, the subject is not ruled out as having Parkinson's disease. In another embodiment, when the expression level of the one or more biomarkers in the subject's blood, plasma, or serum sample is statistically similar to the expression level of the corresponding one or more biomarkers obtained from a group of individuals in the statistical sample who have been diagnosed with Parkinson's disease, the subject is not ruled out as having Parkinson's disease. In some embodiments, the expression level of the one or more biomarkers measured in the subject's sample is compared to the average expression level of the corresponding biomarkers in the statistical sample for each group of individuals in the statistical sample.

In some embodiments, the method further comprises referring the subjects who are not ruled out as having Parkinson's disease to a PD specialist for diagnostic testing.

In some embodiments, the step of measuring the expression level of the one or more biomarkers in the blood, plasma, or serum sample comprises measuring the expression level of each of tenacin C; tenacin C and IL-6; tenacin C, IL-6, and 1309; tenacin C, IL-6, 1309, and IL-7; tenacin C, IL-6, 1309, IL-7, and FABP3; tenacin C, IL-6, 1309, IL-7, FABP3, and TARC; tenacin C, IL-6, 1309, IL-7, FABP3, TARC, and TNFα; tenacin C, IL-6, 1309, IL-7, FABP3, TARC, TNFα, and A2M; of tenacin C, IL-6, 1309, IL-7, FABP3, TARC, TNFα, A2M, and TPO; tenacin C, IL-6, 1309, IL-7, FABP3, TARC, TNFα, and A2M; tenacin C, IL-6, 1309, IL-7, FABP3, TARC, TNFα, A2M, TPO, and B2M; tenacin C, IL-6, 1309, IL-7, FABP3, TARC, TNFα, A2M, TPO, B2M, and IL-5; tenacin C, IL-6, 1309, IL-7, FABP3, TARC, TNFα, A2M, TPO, B2M, IL-5, and Factor VII; tenacin C, IL-6, 1309, IL-7, FABP3, TARC, TNFα, A2M, TPO, B2M, IL-5, Factor VII, and IL-10; tenacin C, IL-6, 1309, IL-7, FABP3, TARC, TNFα, A2M, TPO, B2M, IL-5, Factor VII, IL-10, and sVCAM-1; tenacin C, IL-6, 1309, IL-7, FABP3, TARC, TNFα, A2M, TPO, B2M, IL-5, Factor VII, IL-10, sVCAM-1, and eotaxin 3; tenacin C, IL-6, 1309, IL-7, FABP3, TARC, TNFα, A2M, TPO, B2M, IL-5, Factor VII, IL-10, sVCAM-1, eotaxin 3, and SAA; tenacin C, IL-6, 1309, IL-7, FABP3, TARC, TNFα, A2M, TPO, B2M, IL-5, Factor VII, IL-10, sVCAM-1, and eotaxin 3; tenacin C, IL-6, 1309, IL-7, FABP3, TARC, TNFα, A2M, TPO, B2M, IL-5, Factor VII, IL-10, sVCAM-1, eotaxin 3, SAA, and IL-18; tenacin C, IL-6, 1309, IL-7, FABP3, TARC, TNFα, A2M, TPO, B2M, IL-5, Factor VII, IL-10, sVCAM-1, and eotaxin 3; tenacin C, IL-6, 1309, IL-7, FABP3, TARC, TNFα, A2M, TPO, B2M, IL-5, Factor VII, IL-10, sVCAM-1, eotaxin 3, SAA, IL-18, and sICAM-1; tenacin C, IL-6, 1309, IL-7, FABP3, TARC, TNFα, A2M, TPO, B2M, IL-5, Factor VII, IL-10, sVCAM-1, eotaxin 3, SAA, IL-18, sICAM-1, and CRP. In some embodiments, the expression level of each of the biomarkers measured above is compared to the expression level of each of the corresponding biomarkers in the statistical sample to determine if the subject is excluded from diagnostic testing for Parkinson's disease.

In certain embodiments, the expression level of one or more of tenacin C, IL-6, 1309, IL-7, FABP3, TARC, TNFα, A2M, TPO, and B2M is measured in the blood, plasma, or serum sample. In certain embodiments, the expression level of each of tenacin C, IL-6, 1309, IL-7, FABP3, TARC, TNFα, A2M, TPO, and B2M is measured in the blood, plasma, or serum sample. In certain embodiments, the expression level of each of tenacin C, IL-6, 1309, and IL-7, and optionally FABP3, is measured in the blood, plasma, or serum sample.

In certain embodiments, the biomarker measurements are obtained by a method selected from the group consisting of an immunoassay, an enzymatic activity assay, fluorescence detection, chemiluminescence detection, electrochemiluminescence detection and patterned array, reverse transcriptase-polymerase chain reaction, antibody binding, fluorescence activated sorting, detectable bead sorting, antibody array, microarray, enzymatic array, receptor binding array, allele specific primer extension, target specific primer extension, solid-phase binding array, liquid phase binding array, fluorescent resonance transfer, and radioactive labeling. In some embodiments, the biomarker measurements are obtained by electrochemiluminescence detection.

In some embodiments, the diagnostic test is selected from the group consisting of MRI, dopamine transporter (DAT) scan, brain ultrasound, PET scan, detailed neuropsychological testing, and any combinations thereof. In some embodiments, the treatment is selected from the group consisting of levodopa, a dopamine agonist, a glutamate agonist, an anticholinergic agent, a catechol-o-methyl transferase (COMT) inhibitor, a monoamine oxidase type B (MAO-B) inhibitor, a dopaminergic therapy, an N-methyl-D-aspartate (NMDA) antagonist, and any combinations thereof.

### Kits

In yet another aspect, the present disclosure relates to a kit for screening a subject to determine whether the subject can be ruled out as having Parkinson's disease.

In certain embodiments, the kit comprises a syringe and needle for use in obtaining a blood, plasma, or serum sample from the subject. In certain embodiments, the kit comprises an instruction booklet.

In certain embodiments, the kit is intended for use in a primary care setting. In certain embodiments, the kit comprises one or more reagents that comprises a detectable marker for use in electrochemiluminescence detection. In some embodiments, the one or more reagents that comprises a detectable marker are adapted for use in a multiplex biomarker assay platform using electrochemiluminescence. In certain embodiments, the detectable marker is used to measure, in a blood, serum, or plasma sample, the expression level of one or more biomarkers described elsewhere herein. In certain embodiments, the kit comprises a “normal” blood, plasma, or serum sample in order to compare the expression level of the one or more biomarkers in the subject's sample to “normal” sample. In certain embodiments, the kit comprises a blood, serum, or plasma sample from an individual who has been diagnosed with PD in order to compare the expression level of the one or more biomarkers from the subject's sample to the PD sample. In another embodiment, the kit comprises an algorithm or a code segment comprising an algorithm to run on a suitable processor. In certain embodiments, the algorithm compares the measured expression level of each biomarker with a statistical sample representative of the subject. In certain embodiments, the statistical sample is described elsewhere herein. In certain embodiments, the kit comprises instructions for how to determine whether the subject can be ruled out as having Parkinson's disease. In another embodiment, the kit comprises a second code segment that can be run on a suitable processor, wherein the second code segment determines whether the subject can be ruled out as having Parkinson's disease.

In yet another aspect, the present disclosure relates to a kit for use as a direct-to-consumer (DTC) product. In certain embodiments the DTC kit comprises instructions that would permit a subject to self-refer to a primary care physician to determine if the subject can be ruled out as having PD. In certain embodiments, the instructions comprise a recitation of common early warning signs of PD that the subject can use to self-refer to a primary care physician. In certain embodiments, a subject who has one or more early warning signs of PD is instructed to visit a primary care physician to determine if the subject can be ruled out as having PD. In certain embodiments, a method disclosed elsewhere herein is used to determine if the subject can be ruled out as having PD.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1: Parkinson's Disease Blood Test for Primary Care

A series of studies have been conducted demonstrating the utility of blood-based biomarkers for detecting PD as well as discriminating PD from other neurodegenerative diseases. However, no studies have been conducted demonstrating the utility of blood-based biomarkers for ruling out PD in a primary care setting. Herein, a large-scale cross-validation of the PD Blood Test (PDBT) for use in primary care settings was completed.

### Methods

### Participants and Reference Database

Parkinson's Disease Data. Baseline and longitudinal assays were completed on serum samples from the previously conducted DATATOP trial. The DATATOP Study was a multi-site placebo-controlled clinical trial designed to test the impact of deprenyl 10 mg/d and/or tocopherol (vitamin E) 2000 IU/d on PD progression (in combination with levodopa). A total of 656 baseline PD serum samples had the requisite data for use in the current study. An additional n=190 serum samples from PD cases were already included in a research database from PD specialty evaluations. Therefore, there was a total number of n=846 PD cases. No cases included in this study had a diagnosis of PD-dementia.

Neurodegenerative Disease Blood Test Reference Database (NDRD). Complex diseases, such as neurodegenerative diseases, require that multiple factors (or biological pathways) be considered when making a diagnosis rather than just a single factor. In prior work, a blood test for detecting AD specifically for use in primary care settings was generated. This blood test was discovered and validated on the premise that taking multiple biomarkers into account would yield a more accurate approach than any single marker. This multi-marker approach has led to multiple in vitro diagnostic (IVD) tests being advanced to clinical use in the field of oncology. However, in order advance such an “algorithm” to the clinic requires an appropriate Reference Database that, in practice, when combined with the algorithm itself would be covered under FDA regulations as Software as a Medical Device (SAMD). Therefore, a NDRD was generated and published (O'Bryant, S. E. et al., “Comparing biological markers of Alzheimer's disease across blood fraction and platforms: Comparing apples to oranges,” Alzheimer's Dement. Diagnosis, Assess. Dis. Monit., 2016, 3:27-34). The NDRD contains data from n>5000 participants across a broad range of diseases (e.g., AD, PD, DLB, controls) and blood fractions (serum and plasma). Only completely de-identified data are included in the NDRD. To be included in the NDRD, the data came from studies that (1) conducted comprehensive cognitive assessments on all participants for accurate diagnosis and (2) were conducted under IRB approval and written informed consent was obtained.

Controls: Controls in the database had no neurodegenerative disease diagnosis, performed within normal cognitive parameters on neuropsychological testing and reported no decline in activities of daily living. For the purpose this study, control samples from serum data were utilized (controls n=2,291).

### Proteomics

All serum samples were assayed using the Hamilton Robotics EasyBlood for blood processing, aliquoting, and re-aliquoting. A custom Hamilton Robotics StarPlus system was utilized for the preparation of all plates. Proteomic assays were run on a multi-plex biomarker assay platform using electrochemiluminescence (ECL) per previously published methods using commercially available kits.

ECL technology uses labels that emit light when electronically stimulated, which improves the sensitivity of detection for many analytes even at very low concentrations. ECL measures have well established properties for being more sensitive and requiring less volume than conventional ELISAs, the gold standard for most assays. The analytic performance of several proteins for n>1,300 samples across multiple cohorts and diagnoses (normal cognition, mild cognitive impairment, and AD) was recently reported. The assays are reliable and show excellent spiked recovery, dilution linearity, coefficients of variation, as well as detection limits. Inter- and intra-assay variability has been excellent. Internal QC protocols are implemented in addition to manufacturing protocols including assaying consistent controls across batches and assay of pooled standards across lots. A total of 500 μl of serum was utilized to assay (singlicate) the following markers: fatty acid binding protein (FABP)-3, beta 2 microglobulin (B2M), pancreatic polypeptide (PPY), c-reactive protein (CRP), ICAM-1, thrombopoietin, α2 macroglobulin (A2M), exotaxin 3, tumor necrosis factor alpha (TNF-α), tenascin C, interleukin (IL)-5, IL-6, IL-7, IL-10, IL-18, I-309, Factor 7 (Factor VII), vascular cell adhesion molecule 1 (VCAM 1), TARC and serum amyloid a (SAA). Over 20,000 of these assays have been run over the last several years with all CVs being <10% with the majority being <=6%.

### Statistical Analysis

Statistical analyses were conducted using the R (V 3.3.3) statistical software, SPSS 24 (IBM), and SAS. Support vector machine (SVM) analyses were conducted to discriminate PD cases from controls. SVM is based on the concept of decision planes that define decision boundaries and is primarily a classifier method that performs classification tasks by constructing hyperplanes in a multidimensional space that separates cases of different class labels. Diagnostic accuracy was calculated via receiver operating characteristic (ROC) curves. The sample was randomly split (70/30) into training and test samples with diagnostic accuracy derived from the test sample. Finally, to provide estimates of the overall utility of the PDBT in ruling out PD in primary care settings, negative predictive values (NPVs; the probability that subjects with a negative screening test truly do not have disease) were calculated using a range of base rates including 2%, 5%, 10%, and 15%.

### Results

Descriptive statistics of the sample are provided in Table 1. The average age of the sample 63.8 (SD=13.4). The PD group was younger, more likely to be male, and reported higher levels of education (p-values <0.001) as compared to the normal control group.

TABLE 1
Demographic characteristics of the cohort

| | PD | | Normal Control | | |
|---|---|---|---|---|---|
| | Mean (SD) | | Mean (SD) | | p-value |
| N | 846 | | 2291 | | |
| Age | 59.5 | (12.6) | 65.4 | (13.4) | 4.16e−29 |
| Education | 13.76 | (4.65) | 12.74 | (6.62) | 1.59e−6 |

| | | | |
|---|---|---|---|
| Gender (% male) | 62.8 | 40.1 | 5.65e−30 |

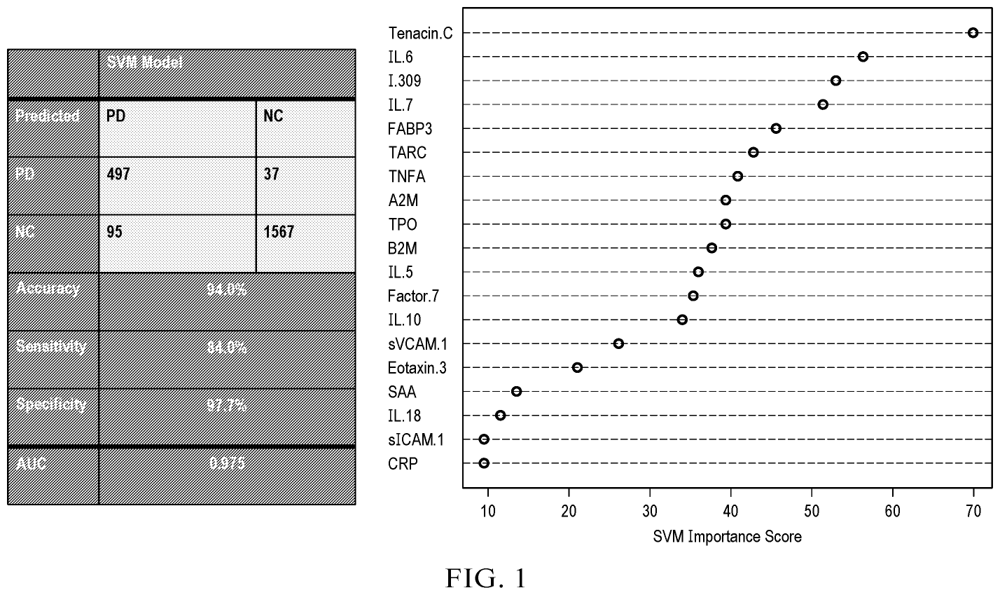

In the training sample, there was a total 592 PD samples and 1604 control samples. The SVM was applied with a 5-fold internal cross-validation within the training sample for initial analysis and internal validation. The PDBT yielded an AUC of 0.98 with a SN of 0.84 and SP of 0.98 within the training set. FIG. 1 shows the overall classification accuracy (correct and incorrect) along with the diagnostic accuracy statistics and variable importance plot.

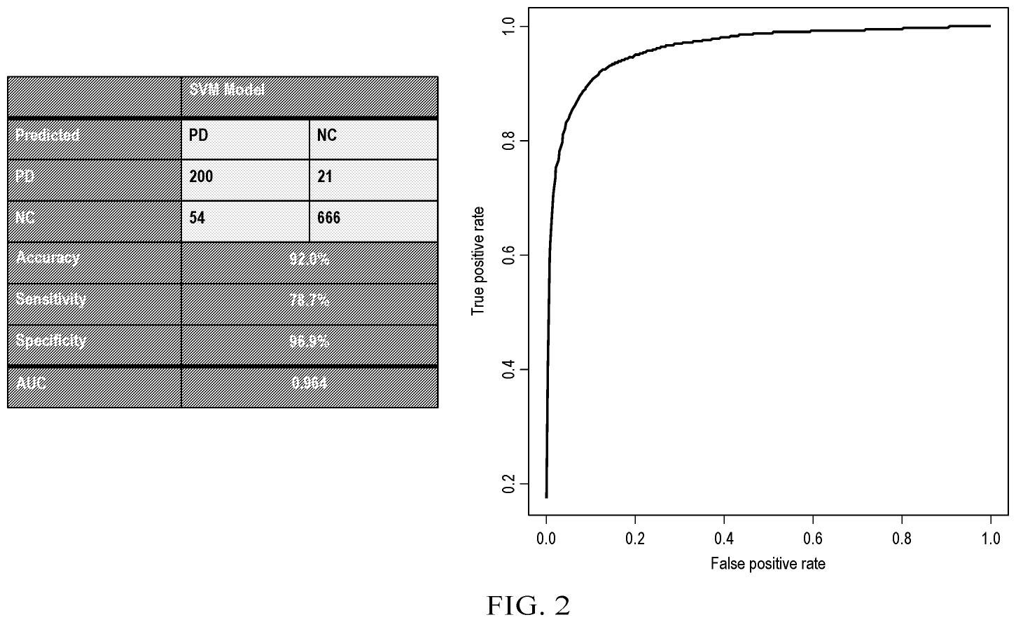

Next, the PDBT was directly applied to the test sample, which consisted of n=254 PD cases and n=687 controls. The PDBT yielded an AUC of 0.964 with a SN of 0.79 and SP of 0.97. FIG. 2 provides the classification accuracy (correct and incorrect) as well as the ROC curve.

Finally, to provide a sense of how the PDBT would perform as a screening tool for ruling out PD in primary care settings, the NPV was calculated for a range of base rates. With a 2% base rate, the NPV was 0.99. Therefore, the physician is 99% accurate in ruling out PD with a negative blood test. The NPV for 5%, 10% and 15% base rates were 99%, 98%, and 96%, respectively. If a physician used a 5% base rate for those adults complaining of new onset motor changes, and saw 5000 patients, the PDBT would rule out 4,660 patients from needing any additional testing procedures. There would be only 53 false negative cases.

### Selected Discussion

The data demonstrate the utility of the PDBT as a screen for ruling out PD in primary care settings. In the current study, data were pooled for an aggregate sample of 846 PD samples and 2,291 control samples. Overall, the accuracy of the PDBT is excellent (i.e., >98%) for ruling out disease. The goal of a screening test in primary care settings for neurodegenerative diseases is to screen out the disease, which is consistent with the use and performance of the vast majority of screening tests used in primary care settings on a daily basis.

The COU for the PDBT is not diagnostic, but rather as a screening tool to rule out PD within primary care settings. The availability of the PDBT for screening purposes in primary care holds tremendous benefit. First, this is a rapidly scalable technology that can be implemented globally as a laboratory developed test (LTD). The PDBT provides primary care providers with actionable and objective information that is supported by several studies and many patients. Additionally, it is thought that the earlier therapeutics can be administered, the more beneficial they are to patients. The availability of the PDBT in primary care settings provides a tool for rapid referrals. Finally, for clinical trials, the PDBT provides a means of drastically expanding access to screening procedures well beyond specialty clinics. Overall, the current results strongly support the utility of the PDBT for the COU of screening out PD in primary care settings.

A person of skill in the art would readily recognize that steps of various above-described methods can be performed by programmed computers. Herein, some embodiments are also intended to cover program storage devices, e.g., digital data storage media, which are machine or computer-readable and encode machine-executable or computer-executable programs of instructions, wherein said instructions perform some or all of the steps of said above-described methods. The program storage devices may be, e.g., digital memories, magnetic storage media such as magnetic disks and magnetic tapes, hard drives, or optically readable digital data storage media. The embodiments are also intended to cover computers programmed to perform said steps of the above-described methods.

The functions of the various elements shown in the figures, including any functional blocks labeled as “modules”, may be provided through the use of dedicated hardware as well as hardware capable of executing software in association with the appropriate software. When provided by a processor, the functions may be provided by a single dedicated processor, by a single shared processor, or by a plurality of individual processors, some of which may be shared. Moreover, explicit use of the term “module” should not be construed to refer exclusively to hardware capable of executing software, and may implicitly include, without limitation, digital signal processor (DSP) hardware, network processor, application-specific integrated circuit (ASIC), field-programmable gate array (FPGA), read-only memory (ROM) for storing software, random access memory (RAM), and nonvolatile storage. Other hardware, conventional and/or custom, may also be included.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

1. A method for excluding a subject from the need for diagnostic testing for Parkinson's disease, the method comprising:
(a) obtaining a blood, plasma, or serum sample from the subject;
(b) measuring in the blood, plasma, or serum sample the expression level of one or more biomarkers selected from the group consisting of: interleukin (IL)-7, tumor necrosis factor alpha (TNFα), IL-5, IL-6, C-reactive protein (CRP), IL-10, soluble intracellular adhesion molecule (sICAM-1), Factor VII, 1309, alpha-2-microglobulin (A2M), Chemokine (C-C Motif) Ligand 17 (TARC), eotaxin 3, soluble vascular cell adhesion molecule 1 (sVCAM-1), thrombopoietin (TPO), fatty acid binding protein (FABP), IL-18, beta-2-microglobulin (B2M), serum amyloid A1 cluster (SAA), tenacin C, tumor necrosis factor receptor 1 (TNFR1), and optionally pancreatic polypeptide (PPY);
(c) using a machine learning algorithm comparing the expression level of the one or more biomarkers in (b) with a statistical sample representative of the subject, thus determining whether the subject can be ruled out as having Parkinson's disease; and
(d) determining that the subject is to be excluded from diagnostic testing for Parkinson's disease based on the comparing step.

2. The method of claim 1, wherein the method further comprises (e) avoiding, not commencing, or discontinuing a diagnostic test for Parkinson's disease, wherein the diagnostic test is selected from the group consisting of neurological examination, MRI, dopamine transporter (DAT) scan, brain ultrasound, PET scan, detailed neuropsychological testing, and any combinations thereof.

3. The method of claim 1, wherein the method further comprises (f) avoiding, not commencing, or discontinuing a treatment for Parkinson's disease, wherein the treatment is selected from the group consisting of levodopa, a dopamine agonist, a glutamate agonist, an anticholinergic agent, a catechol-o-methyl transferase (COMT) inhibitor, a monoamine oxidase type B (MAO-B) inhibitor, a dopaminergic therapy, an N-methyl-D-aspartate (NMDA) antagonist, and any combinations thereof.

4. The method of claim 1, wherein the one or more biomarkers are selected from the group consisting of tenacin C, IL-6, 1309, IL-7, and FABP;
the expression level of each biomarker in the group consisting of tenacin C, IL-6, 1309, and IL-7 is measured; and optionally the expression level of one or more biomarkers selected from the group consisting of FABP, TNFα, IL-5, CRP, IL-10, sICAM-1, Factor VII, 1309, A2M, TARC, eotaxin 3, sVCAM-1, TPO, IL-18, B2M, SAA, and PPY is measured: or
the expression level of the one or more biomarkers is measured using electrochemiluminescence.

5. (canceled)

6. (canceled)

7. The method of claim 1, wherein
(i) when the expression level of the one or more biomarkers in (b) is statistically similar to the average expression level of the corresponding one or more biomarkers obtained from a group of individuals in the statistical sample who do not have Parkinson's disease, the subject is excluded from diagnostic testing for Parkinson's disease; or
(ii) when the expression level of the one or more biomarkers in (b) is not statistically similar to the average expression level of the corresponding one or more biomarkers obtained from a group of individuals in the statistical sample who have been diagnosed with Parkinson's disease, the subject is excluded from diagnostic testing for Parkinson's disease.

8. A method for excluding a subject from recruitment into a clinical trial for an investigational Parkinson's disease medication, the method comprising:
(a) obtaining a blood, plasma, or serum sample from the subject;
(b) measuring in the blood, plasma, or serum sample the expression level of one or more biomarkers selected from the group consisting of: interleukin (IL)-7, tumor necrosis factor alpha (TNFα), IL-5, IL-6, C-reactive protein (CRP), IL-10, soluble intracellular adhesion molecule (sICAM-1), Factor VII, 1309, alpha-2-microglobulin (A2M), Chemokine (C-C Motif) Ligand 17 (TARC), eotaxin 3, soluble vascular cell adhesion molecule 1 (sVCAM-1), thrombopoietin (TPO), fatty acid binding protein (FABP), IL-18, beta-2-microglobulin (B2M), serum amyloid A1 cluster (SAA), tenacin C, tumor necrosis factor receptor 1 (TNFR1), and optionally pancreatic polypeptide (PPY);
(c) using a machine learning algorithm comparing the expression level of the one or more biomarkers in (b) with a statistical sample representative of the subject; and
(d) excluding the subject from recruitment into the clinical trial if the subject is ruled out of having Parkinson's disease from the comparison with the statistical sample.

9. The method of claim 8, the method further comprising:
(e) avoiding, not commencing, or discontinuing a diagnostic test for Parkinson's disease, wherein the diagnostic test is selected from neurological examination, MRI, dopamine transporter (DAT) scan, brain ultrasound, PET scan, and detailed neuropsychological testing.

10. The method of claim 8, the method further comprising:
(f) avoiding, not commencing, or discontinuing a treatment for Parkinson's disease, wherein the treatment is selected from levodopa, a dopamine agonist, a glutamate agonist, an anticholinergic agent, a catechol-o-methyl transferase (COMT) inhibitor, a monoamine oxidase type B (MAO-B) inhibitor, a dopaminergic therapy, an N-methyl-D-aspartate (NMDA) antagonist, and any combinations thereof.

11. The method of claim 8, wherein the one or more biomarkers are selected from the group consisting of tenacin C, IL-6, 1309, IL-7, and FABP;
the expression level of each biomarker in the group consisting of tenacin C, IL-6, 1309, and IL-7 is measured; and optionally the expression level of one or more biomarkers selected from the group consisting of FABP, TNFα, IL-5, CRP, IL-10, sICAM-1, Factor VII, 1309, A2M, TARC, eotaxin 3, sVCAM-1, TPO, IL-18, B2M, SAA, and PPY is measured: or
the expression level of the one or more biomarkers is measured using electrochemiluminescence.

12. (canceled)

13. (canceled)

14. The method of claim 8, wherein
(i) when the expression level of the one or more biomarkers in (b) is statistically similar to the average expression level of the corresponding one or more biomarkers obtained from a group of individuals in the statistical sample who do not have Parkinson's disease, the subject is excluded from recruitment into the clinical study; or
(ii) the expression level of the one or more biomarkers in (b) is not statistically similar to the average expression level of the corresponding one or more biomarkers obtained from a group of individuals in the statistical sample who have been diagnosed with Parkinson's disease, the subject is excluded from recruitment into the clinical study.

15. A method for screening a subject to determine whether the subject is ruled out as having Parkinson's disease, the method comprising:
(a) obtaining a blood, plasma, or serum sample from the subject;
(b) measuring in the blood, plasma, or serum sample the expression level of one or more biomarkers selected from the group consisting of: interleukin (IL)-7, tumor necrosis factor alpha (TNFα), IL-5, IL-6, C-reactive protein (CRP), IL-10, soluble intracellular adhesion molecule (sICAM-1), Factor VII, 1309, alpha-2-microglobulin (A2M), Chemokine (C-C Motif) Ligand 17 (TARC), eotaxin 3, soluble vascular cell adhesion molecule 1 (sVCAM-1), thrombopoietin (TPO), fatty acid binding protein (FABP), IL-18, beta-2-microglobulin (B2M), serum amyloid A1 cluster (SAA), tenacin C, tumor necrosis factor receptor 1 (TNFR1), and optionally pancreatic polypeptide (PPY);
(c) using a machine learning algorithm comparing the expression level of the one or more biomarkers in (b) with a statistical sample representative of the subject, thus determining whether the subject is ruled out as having Parkinson's disease; and
(d) excluding the subjects who are ruled out as having Parkinson's disease from a diagnostic test for Parkinson's disease, a treatment of Parkinson's disease, or a combination thereof; or
(e) administering a diagnostic test for Parkinson's disease, a treatment for Parkinson's disease, or a combination thereof to the subjects who are not ruled out as having Parkinson's disease.

16. The method of claim 15, wherein the diagnostic test is selected from the group consisting of neurological examination, MRI, dopamine transporter (DAT) scan, brain ultrasound, PET scan, detailed neuropsychological testing, and any combinations thereof.

17. The method of claim 15, wherein the treatment is selected from the group consisting of levodopa, a dopamine agonist, a glutamate agonist, an anticholinergic agent, a catechol-o-methyl transferase (COMT) inhibitor, a monoamine oxidase type B (MAO-B) inhibitor, a dopaminergic therapy, an N-methyl-D-aspartate (NMDA) antagonist, and any combinations thereof.

18. The method of claim 15, wherein the one or more biomarkers are selected from the group consisting of tenacin C, IL-6, 1309, IL-7, and FABP;
the expression level of each biomarker in the group consisting of tenacin C, IL-6, 1309, and IL-7 is measured; and optionally the expression level of one or more biomarkers selected from the group consisting of FABP, TNFα, IL-5, CRP, IL-10, sICAM-1, Factor VII, 1309, A2M, TARC, eotaxin 3, sVCAM-1, TPO, IL-18, B2M, SAA, and PPY is measured: or
the expression level of the one or more biomarkers is measured using electrochemiluminescence.

19. (canceled)

20. (canceled)

21. The method of claim 15, wherein
(i) when the expression level of the one or more biomarkers in (b) is not statistically similar to the average expression level of the corresponding one or more biomarkers obtained from a group of individuals in the statistical sample who do not have Parkinson's disease, the subject is not ruled out as having Parkinson's disease; or
(ii) when the expression level of the one or more biomarkers in (b) is statistically similar to the average expression level of the corresponding one or more biomarkers obtained from a group of individuals in the statistical sample who have been diagnosed with Parkinson's disease, the subject is not ruled out as having Parkinson's disease.

22. The method of claim 18, wherein the method further comprises (f) referring the subjects not ruled out as having Parkinson's disease to a specialist in Parkinson's disease.

23. A method for excluding a subject from the need for diagnostic testing for Parkinson's disease, the method comprising:
(a) obtaining a blood, plasma, or serum sample from the subject;
(b) measuring in the blood, plasma, or serum sample the expression level of one or more biomarkers selected from the group consisting of: interleukin (IL)-7, tumor necrosis factor alpha (TNFα), IL-5, IL-6, C-reactive protein (CRP), IL-10, soluble intracellular adhesion molecule (sICAM-1), Factor VII, 1309, alpha-2-microglobulin (A2M), Chemokine (C-C Motif) Ligand 17 (TARC), eotaxin 3, soluble vascular cell adhesion molecule 1 (sVCAM-1), thrombopoietin (TPO), fatty acid binding protein (FABP), IL-18, beta-2-microglobulin (B2M), serum amyloid A1 cluster (SAA), tenacin C, tumor necrosis factor receptor 1 (TNFR1), and optionally pancreatic polypeptide (PPY);
(c) using a machine learning algorithm comparing, using a computer, the expression level of the one or more biomarkers in (b) with a statistical sample representative of the subject, thus determining whether the subject can be ruled out as having Parkinson's disease; and
(d) determining, using a computer, that the subject is to be excluded from diagnostic testing for Parkinson's disease based on the comparing step.

24. The method of claim 23, wherein the method further comprises (e) avoiding, not commencing, or discontinuing a diagnostic test for Parkinson's disease, wherein the diagnostic test is selected from the group consisting of neurological examination, MRI, dopamine transporter (DAT) scan, brain ultrasound, PET scan, detailed neuropsychological testing, and any combinations thereof.

25. The method of claim 23, wherein the method further comprises (f) avoiding, not commencing, or discontinuing a treatment for Parkinson's disease, wherein the treatment is selected from the group consisting of levodopa, a dopamine agonist, a glutamate agonist, an anticholinergic agent, a catechol-o-methyl transferase (COMT) inhibitor, a monoamine oxidase type B (MAO-B) inhibitor, a dopaminergic therapy, an N-methyl-D-aspartate (NMDA) antagonist, and any combinations thereof.

26. The method of claim 23, wherein the one or more biomarkers are selected from the group consisting of tenacin C, IL-6, 1309, IL-7, and FABP;
the expression level of each biomarker in the group consisting of tenacin C, IL-6, 1309, and IL-7 is measured; and optionally the expression level of one or more biomarkers selected from the group consisting of FABP, TNFα, IL-5, CRP, IL-10, sICAM-1, Factor VII, 1309, A2M, TARC, eotaxin 3, sVCAM-1, TPO, IL-18, B2M, SAA, and PPY is measured: or
the expression level of the one or more biomarkers is measured using electrochemiluminescence.

27. (canceled)

28. (canceled)

29. The method of claim 23, wherein
(i) when the expression level of the one or more biomarkers in (b) is determined by a computer to be statistically similar to the average expression level of the corresponding one or more biomarkers obtained from a group of individuals in the statistical sample who do not have Parkinson's disease, the subject is excluded from diagnostic testing for Parkinson's disease; or
(ii) when the expression level of the one or more biomarkers in (b) is determined by a computer that is not statistically similar to the average expression level of the corresponding one or more biomarkers obtained from a group of individuals in the statistical sample who have been diagnosed with Parkinson's disease, the subject is excluded from diagnostic testing for Parkinson's disease.
